(19) United States
(12) Patent Application Publication
(10) Pub. No.: US 2025/0352192 A1
(43) Pub. Date: Nov. 20, 2025

(54) DELIVERY SYSTEMS, DEVICES, ASSEMBLIES, AND METHODS FOR HEMOSTATIC OR ADHESIVE AGENTS

(71) Applicant: Boston Scientific Scimed, Inc., Maple Grove, MN (US)

(72) Inventors: Rosangel Aimee RAMOS ESPINOZA, Boston, MA (US); Deocraciano Aristocrascio CARDOSO, Marlborough, MA (US); Ryan V. WALES, Shrewsbury, MA (US)

(73) Assignee: Boston Scientific Scimed, Inc., Maple Grove, MN (US)

(21) Appl. No.: 19/203,850

(22) Filed: May 9, 2025

Related U.S. Application Data
(60) Provisional application No. 63/647,729, filed on May 15, 2024.

(51) Int. Cl. A61B 17/00

(52) U.S. Cl. CPC A61B 17/00491; A61B 2017/0034; A61B 2017/00495

(57) ABSTRACT

Systems, devices, and related methods for delivering substances to a target treatment site within a subject (e.g., patient) are described. The medical device includes a shaft including a plurality of lumens, each of the plurality of lumens including an inlet at a proximal portion of the shaft and an outlet at a distal portion of the shaft. A mixer assembly is disposed in the distal portion of the shaft and in fluid communication with the plurality of lumens. The mixer assembly includes a mixing chamber configured to receive a plurality of substances from the plurality of lumens, a piston movably disposed in the mixing chamber, and a driver operably coupled to the piston. The driver is configured to drive the piston relative to the shaft within the mixing chamber, and in turn expel the plurality of substances from the mixing chamber.

### CROSS-REFERENCE TO RELATED APPLICATIONS

This application claims the benefit of priority of U.S. Provisional Patent Application No. 63/647,729, filed on May 15, 2024, the entirety of which is incorporated herein by reference.

### TECHNICAL FIELD

Various aspects of this disclosure relate generally to medical delivery systems, devices, assemblies, and related methods for delivering hemostatic or adhesive agents. For example, the disclosure includes systems, devices, assemblies, and related methods for delivering a gel or fluid mixture to a target treatment site of a subject.

### BACKGROUND

During medical procedures, such as endoscopic procedures, one or more substances (e.g., treatment substances) may be delivered to a target treatment site. For instance, during an endoscopic procedure, a user inserts a shaft of an endoscope into a body lumen of a patient. The user operates a handle of the endoscope to control (e.g., deflect and/or position) the endoscope during the procedure. The user may pass one or more tools through a working channel of the endoscope (e.g., via a port in the handle) to deliver treatment at the target treatment site (e.g., internal to the patient) near a distal end of the endoscope. Treatment may include delivering one or more substances at the target treatment site through the working channel and/or through a delivery device inserted within the working channel. Several substances used in medical procedures (e.g., treatment substances, such as cyanoacrylates or other bonding substances, adhesives, etc.) may cure or harden within the delivery device and/or the working channel, potentially forming clogs or obstructions therein. Similarly, two or more substances may be mixed to form a mixture of substances for use during the medical procedure; however, the mixture of substances may cure or harden within the delivery device and/or working channel before being delivered to the target treatment site. These clogs or obstructions may prevent or inhibit delivering the substance(s) through the delivery device and/or working channel, which may increase the duration, costs, and/or risks associated with the medical procedure.

### SUMMARY

Each of the aspects disclosed herein may include one or more of the features described in connection with any of the other disclosed aspects.

According to an example, a medical device may include a shaft and a mixer assembly. The shaft may include a plurality of lumens. Each of the plurality of lumens may include an inlet at a proximal portion of the shaft and an outlet at a distal portion of the shaft. The mixer assembly may be disposed in the distal portion of the shaft and may be in fluid communication with the plurality of lumens. The mixer assembly may include a mixing chamber configured to receive a plurality of substances from the plurality of lumens. The mixer assembly may also include a piston and a driver operably coupled to the piston. The piston may be movably disposed in the mixing chamber. The driver may be configured to drive the piston relative to the shaft within the mixing chamber, and in turn expel the plurality of substances from the mixing chamber.

Any medical device or medical system described herein may include any of the following features. The mixer assembly may include a distal tip in fluid communication with a distal end of the mixing chamber. The distal tip may be a duckbill nozzle configured to expel the plurality of substances through an opening formed therein. The mixing chamber may include an outer surface having an external thread disposed thereon, and the distal tip may include an internal thread configured to threadably engage the external thread. The driver may include a movable portion configured to move within the mixing chamber, and the driver may also include an arm coupled to the movable portion and the piston. The mixer assembly may include a plurality of ports in fluid communication with the plurality of lumens and the mixing chamber. The plurality of ports may include a first port extending into the mixing chamber at a first location relative to a longitudinal axis of the mixing chamber. The plurality of ports may also include a second port extending into the mixing chamber at a second location relative to the longitudinal axis. The first location may be positioned proximal of the second location and distal of the movable portion of the driver relative to the longitudinal axis. The first location may be positioned proximal of a distalmost position of the piston, and the second location may be positioned distal of a proximalmost position of the piston.

The mixer assembly may include a transfer conduit including a first end in fluid communication with a third location of the mixing chamber, and the mixer assembly may also include a second end in fluid communication with a fourth location of the mixing chamber. The third location may be positioned adjacent to the movable portion of the driver and proximal of the first location relative to the longitudinal axis. The fourth location may be positioned distal of the first location and proximal of the second location relative to the longitudinal axis. The movable portion of the driver may include at least one groove configured to displace a volume of at least one substance of the plurality of substances from the mixing chamber toward the first end of the transfer conduit. The movable portion may be configured to move within the mixing chamber about a pivot point, which in turn translates the arm proximally and/or distally relative to the longitudinal axis, and in turn translates the piston longitudinally proximally and/or distally within the mixing chamber along the longitudinal axis.

The driver may include a motor coupled to the movable portion, and the motor may be configured to rotate the movable portion about the pivot point. The motor may include a two-stroke motor. The plurality of lumens may include a first lumen and a second lumen. The plurality of substances that the first lumen and the second lumen are configured to deliver may include a liquid or gel substance and an accelerant substance different from the liquid or gel substance. The first lumen may be configured to deliver the liquid or gel substance into the mixing chamber, and the second lumen may be configured to deliver the accelerant substance into the mixing chamber. The first lumen and the second lumen may be fluidly isolated from each other between a proximal end of the shaft and the mixer assembly.

According to another example, a medical system may include a delivery assembly configured to deliver two or more substances; and a medical device in fluid communication with the delivery assembly. The medical device may include a shaft including a plurality of lumens. Each lumen of the plurality of lumens may include an inlet at a proximal portion of the shaft and a distal opening at a distal portion of the shaft. The medical device may also include a mixer assembly disposed in the distal portion of the shaft and in fluid communication with the plurality of lumens. The mixer assembly may include a mixing chamber configured to receive a plurality of substances from the delivery assembly through the plurality of lumens. The mixer assembly may also include a piston movably disposed in the mixing chamber. and the mixer assembly may further include a driver including a movable portion coupled to the piston The movable portion may be configured to drive the piston relative to the shaft within the mixing chamber, and in turn expel the plurality of substances from the mixing chamber.

Any medical device or medical system described herein may include any of the following features. A controller may be electrically coupled to the mixer assembly. The controller may be configured to control motion of the driver relative to the mixing chamber, and thereby to control motion of the piston through the mixing chamber. The movable portion may include an outer surface having at least one groove formed therein. The groove may be configured to receive a volume of at least one of the plurality of substances therein, and in turn to displace the volume as the movable portion moves relative to the mixing chamber.

According to another example, a medical system may include a first syringe configured to contain and provide a first substance, a second syringe configured to contain and provide a second substance. The medical system may also include a shaft including a first lumen in fluid communication with the first syringe, a second lumen in fluid communication with the second syringe, and a nozzle at a distal end of the shaft. and the medical system may also include a mixer assembly disposed in a distal portion of the shaft. The mixer assembly may include a mixing chamber in fluid communication with the first lumen and the second lumen. The mixing chamber may be fluidly connected to the nozzle. The mixer assembly may include a piston movably disposed in the mixing chamber. The mixer assembly may also include a driver coupled to the piston. The driver may include a motor configured to move the piston through at least a portion of the mixing chamber from a first position in a proximal portion to a second position at a distal portion.

Any medical device or medical system described herein may include any of the following features. The mixer assembly may include a first port in fluid communication with the mixing chamber and a distal opening of the first lumen. The mixer assembly may also include a second port in fluid communication with the mixing chamber and a distal opening of the second lumen. The mixer assembly may further include a transfer conduit. The transfer conduit may include a proximal opening adjacent to a movable portion of the driver. The transfer conduit may include a distal opening spaced distally from the proximal opening. The movable portion of the driver may be configured to displace a volume of at least one of the first substance and the second substance through the proximal opening of the transfer conduit. The distal opening of the transfer conduit may be positioned across from the first port and the second port relative to a longitudinal axis of the mixing chamber. The distal opening may be positioned distal of at least one of the first port and the second port.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a block diagram of a medical system including a medical device, according to aspects of the disclosure.

FIG. 2 is a lateral cross-section view of a distal portion of the medical device of FIG. 1, according to aspects of this disclosure.

FIG. 3 is a side view of the medical device of FIG. 1, according to aspects of this disclosure.

FIG. 4 is a side view of another medical device, according to aspects of this disclosure.

FIG. 5 is a block diagram of another medical system including another medical device with a mixer assembly, according to aspects of the disclosure.

FIG. 6 is a lateral cross-section view of a distal portion of the medical device of FIG. 5, according to aspects of this disclosure.

FIG. 7 is a perspective view of a distal tip of the medical device of FIG. 5, according to aspects of this disclosure.

FIG. 8 a longitudinal cross-section view of the mixer assembly of the medical device of FIG. 5, according to aspects of this disclosure.

### DETAILED DESCRIPTION

Medical delivery devices with features for facilitating a mixture and application of multiple substances (e.g., two or more) to a target treatment site are included herein. For example, the substances may be applied to the target treatment site to form one or more protective coverings or layers, which may help to prevent bleeding and/or help one or more portions of the target treatment site to heal. A target treatment site for receiving the protective covering may include a tissue wall, such as a portion of an esophagus or other part of the gastrointestinal system of the patient. The devices herein may include features for delivering multiple substances to a distal portion of a medical device and mixing the substances in the distal portion of the medical device. The devices may also deliver the mixed substances distally and/or apply the mixed substances to the target treatment site, for example, to help form the protective covering.

Examples of the disclosure include systems, devices, and methods for delivering substances to a target treatment site within a subject (e.g., patient). In examples, accessing a patient's esophagus includes endoluminal placement of the medical device into the target treatment site. Placement of the medical device may be via a catheter, scope (endoscope, bronchoscope, colonoscope, gastroscope, etc.), tube, or sheath, inserted into an anatomical passageway via a natural orifice or via laparoscopy. The orifice can be, for example, the nose, mouth, or anus, and the placement can be in any portion of the GI tract, including the esophagus, stomach, duodenum, large intestine, or small intestine. Placement also can be in other organs or other bodily spaces reachable via the GI tract, other body lumens, or openings in the body, including via laparoscopy. This disclosure is not limited to any particular medical procedure or target treatment site within a body.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. Proximal and distal directions are labeled with arrows marked “P” and “D”, respectively, throughout various figures. As used herein, the terms “comprises,” “comprising,” or any other variation thereof, are intended to cover a non-exclusive inclusion, such that a process, method, article, or container that comprises a list of elements does not necessarily include only those elements, but may include other elements not expressly listed or inherent to such process, method, article, or container. The term “exemplary” is used in the sense of “example,” rather than “ideal.” As used herein, the terms “about,” “substantially,” and “approximately,” indicate a range of values within +/−5% of a stated value.

Examples of the disclosure may relate to systems, devices, and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). As mentioned above, this disclosure is not limited to any specific medical system, device, assembly, or method, and aspects of the disclosure may be used in connection with any suitable medical system, medical tool or device, and/or medical method, at any suitable site within the body. Various examples described herein include single-use or disposable medical devices.

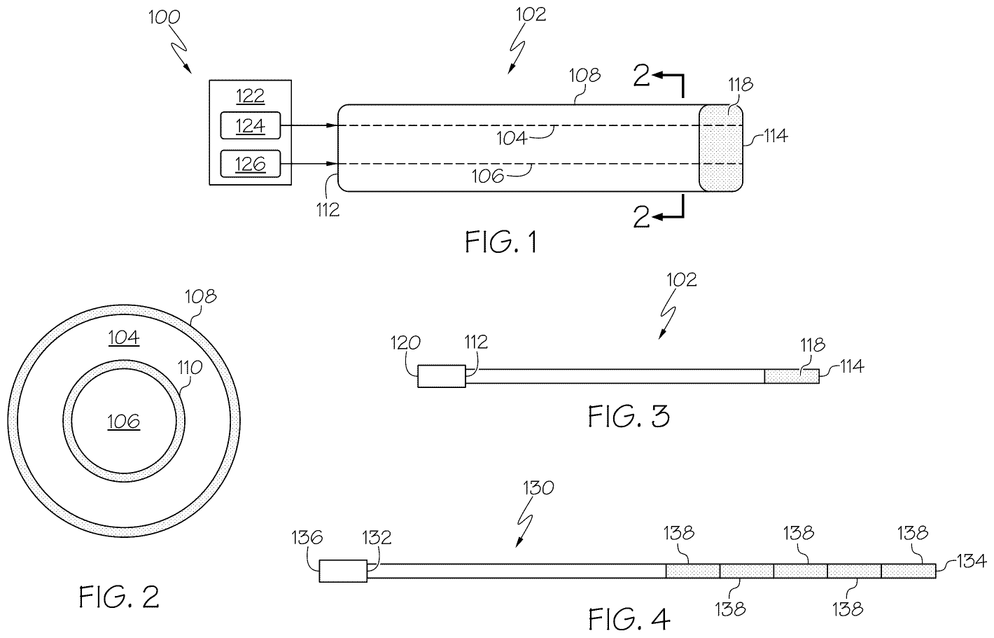

FIG. 1 depicts a medical system 100 in accordance with aspects of the disclosure. The medical system 100 may include a delivery assembly 122 in fluid communication with a shaft 102 (e.g., a catheter). The delivery assembly 122 may be fluidly coupled, directly or indirectly, to shaft 102 using any component or combination of components known in the field. Delivery assembly 122 may transmit one or more substances (e.g., therapeutic substances or agents) through one or more lumens of shaft 102. Delivery assembly 122 may provide two or more substances, which in turn may be combined or mixed in shaft 102 to yield a mixture of substances. The shaft 102 may include two or more lumens, for example, a first lumen 104 and a second lumen 106, and the first lumen 104 and the second lumen 106 may extend from a proximal end 112 of shaft 102 toward a distal end 114 of shaft 102. In some aspects, one or more of first lumen 104 and second lumen 106 may terminate proximal of the distal end 114, for example, such that the substances conveyed by the first lumen 104 and the second lumen 106 may mix within a distal portion of the shaft 102. The mixture of substances may be applied to the target treatment site.

The delivery assembly 122 may have two or more containers. For example, as shown, the delivery assembly 122 may include a first container 124 and a second container 126. The first container 124 and second container 126 may have similar or different characteristics, for example, shapes, sizes, or the like. The first container 124 and second container 126 may be formed of any material capable of containing substance(s) and/or delivering substances (e.g., materials chemically inert to substances contained therein). For example, the first container 124 and the second container 126 may both be syringes. The first container 124 and second container 126 may respectively contain one or more substances therein (e.g., in the form of a gas, fluid, and/or solid (e.g., a powder)). For example, the first container 124 may include a syringe containing a first substance therein, and the second container 126 may include a syringe containing a second substance therein. While only two containers are shown, it should be understood that delivery assembly 122 may include any number of container as appropriate, based on the number of substances, solvents, etc., being delivered to the target treatment site via the shaft 102.

As further shown, the first container 124 may be in fluid communication with the first lumen 104 of the shaft 102, such that the first container 124 may transmit the first substance through the first lumen 104 toward the distal end 114 of the shaft 102. The second container 126 may be in fluid communication with the second lumen 106 of the shaft 102, such that the second container 126 may transmit the second substance through the second lumen 106 toward the distal end 114 of the shaft 102. Each of the first lumen 104 and second lumen 106 may have an inlet (or plurality of inlets) at a proximal portion, and an outlet (or plurality of outlets) at a distal portion. The proximal portion of the first lumen 104 is fluidly isolated from the proximal portion of the second lumen 106. Fluid isolation of the first lumen 104 and the second lumen 106 may help to allow the first container 124 and the second container 126 to respectively transmit the first substance and second substance in fluid isolation through the proximal end 112 of shaft 102. The delivery assembly 122 may distally urge the first substance and second substance through the first lumen 104 and the second lumen 106, respectively, toward the distal end 114 of the shaft 102. The first substance and the second substance may mix in the distal portion of the shaft 102 between the proximal end 112 and the distal end 114, yielding a mixture of substances. The mixture of substances may be distally urged through the shaft 102 toward the distal end 114 and through one or more openings formed therein (not shown).

As discussed herein, the substances may be distally advanced through a mixer 118 disposed in the shaft 102 and proximate to the distal end 114. The mixer 118 may receive both the first substance and the second substance, and may help to mix the first substance and the second substance. In some examples, the mixer 118 may be a static mixer. For instance, in some examples, the mixer 118 may include protrusions or threaded surfaces, which may help to cause substances therein to be mixed during dispersal. In other examples, the mixer 118 may be movable, for example, to introduce energy into the substances flowing through the distal portion of the shaft 102, which may help to mix and/or to activate additional properties of the mixture (e.g., adhesive properties) prior to delivery through the distal end 114 of the shaft 102 to the target treatment site. For instance, in some examples, the mixer 118 may include an auger for mixing two or more substances and advancing the mixture of substances through one or more openings formed in the distal end 114. In some aspects, the mixer 118 may include a single helical thread having a variable diameter and/or a variable pitch. For instance, the variable diameter and/or pitch of the helical thread may decrease at a tapered portion of the distal tip, which may increase distribution precision of one or more substances through the distal end 114 to the target treatment site. However, it should be understood that mixer 118 is not limited to a single thread, thread size, thread pitch, and/or thread surface.

FIG. 2 shows a cross-section view of the shaft 102 along a line 2-2 shown in FIG. 1, according to aspects of the disclosure. As mentioned, the shaft 102 (e.g., flexible sheath, catheter, tube, or the like) may include the first lumen 104 and the second lumen 106. The first lumen 104 and second lumen 106 may be fluidly separated from each other in at least a portion of the shaft 102 (e.g., a proximal portion of the shaft 102). In some aspects, the first lumen 104 and the second lumen 106 are concentric. For example, as shown in FIG. 2, the first lumen 104 is defined by an outer wall 108 of the shaft 102, and surrounds the second lumen 106. The second lumen 106 is defined by an inner wall 110 disposed within the first lumen 104. The inner wall 110 may be attached to the outer wall 108 by one or more protrusions, or other structures, to maintain proper spacing between and position of the second lumen 106 relative to the first lumen 104. The first lumen 104 and second lumen 106 may converge in a distal portion of the shaft 102 (e.g., proximate to the distal end 114 of the shaft 102). For example, the inner wall 110 may terminate in the distal portion of the shaft 102, proximate of the mixer 118. As such, the first substance and second substance may be fluidly isolated in the proximal portion of the shaft 102, and the first substance and the second substance may then mix in the distal portion of the 102 via the mixer 118 prior to delivering the mixture of substances to the target treatment site.

FIG. 3 shows a side view of shaft 102 shown in FIG. 1, according to aspects of the disclosure. As shown, shaft 102 includes the mixer 118 disposed between the proximal end 112 and distal end 114, e.g., disposed in a distal portion of the shaft 102 adjacent the distal end 114. The shaft 102 may include a connector 120 (e.g., luer lock, seal, etc.) to fluidly couple the proximal end 112 and the delivery assembly 122 (FIG. 1) and components thereof (e.g., the first container 124 and/or the second container 126). The connector 120 may include any component to fluidly couple the shaft 102 and delivery assembly 122, for example, such that the first container 124 is fluidly connected to the first lumen 104 and the second container 126 is fluidly connected to the second lumen 106. For instance, the connector 120 may include an internal thread shaped and dimensioned to threadably engage an external thread disposed on or adjacent the proximal end 112 of shaft 102. As such, when the medical system 100 is assembled for use, delivery assembly 122 may transmit the first substance and the second substance through the connector 120 into the first lumen 104 and second lumen 106, respectively, and which in turn converge in the distal portion of the shaft 102, such that the first and second substances flow through the mixer 118 prior to delivery of the mixture of substances at the target treatment site.

FIG. 4 shows a shaft 130, according to another aspect of the disclosure. Like the shaft 102, the shaft 130 may include a proximal end 132 in fluid communication with a delivery assembly, such as the first container 124 of the delivery assembly 122 (FIG. 1). The shaft 130 includes at least one lumen (e.g., two lumens, as discussed above) extending from the proximal end 132 toward a distal end 134. The shaft 130 may include a connector 136 fluidly coupling at least one lumen of the shaft 130 with the delivery assembly 122. The connector 136 may be substantially similar or identical to the connector 120 (FIG. 3), the details of which are omitted herein for brevity. The shaft 130 may further include a plurality of mixers 138 positioned proximate to the distal end 134. The plurality of mixers 138 may introduce energy into at least one substance flowing through a distal portion of the shaft 130, which may activate additional properties the substance(s) towards the distal end 134, such as adhesive properties.

In some aspects, the plurality of mixers 138 may span at least ten percent (10%) of a length of the shaft 130. Each mixer of the plurality of mixers 138 may be substantially similar or identical to the mixer 118 (FIG. 1), the details of which are omitted herein for brevity. In the embodiment shown in FIG. 4, the plurality of mixers 138 includes five (5) mixers in fluid communication with at least one lumen extending in the shaft 130. However, it should be understood that any number of mixers may be disposed within the shaft 130 between the proximal end 132 and distal end 134 thereof. For instance, the shaft 130 may include two (2), three (3), four (4), six (6), or more mixers in the plurality of mixers 138 aligned within the shaft 130 and proximate of distal end 134. Moreover, as shown in FIG. 4, the shaft 130 includes a single lumen in fluid communication with the plurality of mixers 138, in which the single lumen may transmit a single substance (e.g., from the delivery assembly 122) through a proximal opening at the proximal end 132 toward the plurality of mixers 138. However, it should be understood that the shaft 130 may include two or more lumens in fluid communication with a mixing lumen, such that the shaft 130 transmits two or more substances through the plurality of mixers 138.

FIG. 5 shows a medical system 200, according to aspects of the disclosure. The medical system 200 may include a delivery assembly 201 in fluid communication with a shaft 202 (e.g., a catheter). The delivery assembly 201 may include one or more containers configured to transmit one or more substances to the shaft 202. For example, delivery assembly 201 may include a plurality of syringes configured to deliver a plurality of substances through a proximal end 222 of the shaft 202. The shaft 202 may include a plurality of lumens extending therein, and which may be in fluid communication with one or more containers of delivery assembly 201. Each lumen may include an inlet or opening

For example, as shown in FIG. 5, the shaft 202 may include a first lumen 206 and a second lumen 208. The first lumen 206 may be in fluid communication with a first container 203 of the delivery assembly 201, and a second lumen 208 may be in fluid communication with a second container 205 of the delivery assembly 201. The first container 203 and second container 205 may be respectively configured to contain, transmit, deliver, or otherwise provide at least one substance. The first container 203 and second container 205 may be substantially similar or identical to first container 124 and second container 126 (FIG. 1), similar details of which are omitted for brevity. For example, the first container 203 may contain a first substance, and the second container 205 may contain a second substance. The first and second substances may be transmitted through the first lumen 206 and the second lumen 208, respectively, at a proximal end 222 of the shaft 202. The first substance may include, for example, a liquid or gel (e.g., a hemostatic gel). The second substance may include, for example, an accelerant. The accelerant may include a catalyst which, when mixed with the first substance, activates additional properties of the mixture, such as adhesive, hemostatic, or other properties.

Additionally, the shaft 202 may also include a third lumen 210 in fluid communication with a third container 207 of the delivery assembly 201. The third container 207 may be configured to contain, transmit, deliver, or otherwise provide at least one substance. For example, the third container 207 may contain a third substance, such as a gas or plurality of gas species, and the third container 207 may transmit the third substance through proximal end 222 of the shaft 202. The third container 207 may be substantially similar or identical to the first container 203 and second container 205, similar details of which are omitted for brevity. For example, the third container 207 may include a syringe fluidly coupled to the third lumen 210. However, similar to other containers discussed herein, the third container 207 may have any shape and dimension (e.g., cylindrical, spherical, etc.), and formed of any material or combination of materials (e.g., a metal alloy, ceramic, etc.), capable of containing the third substance therein. For instance, the third container 207 may include a gas tank configured to contain oxygen therein. It should be understood that oxygen is a non-limiting example, and that other gas or combinations of gas species may additionally or alternatively be used. The third container 207 may include one or more pressure regulators to maintain pressure of gas therein, and/or to regulate release of gas from the third container 207 through the shaft 202. In some implementations, modifying gas release from third container 207 may help to change a mixture ratio of the fluids, solids, or substances contained in the first container 203 and/or second container 205, for example, when delivered to the target treatment site. Alternatively or additionally, modifying gas release from the third container 207 may help to change the transmission rate of the first substance and/or second substance from the delivery assembly 201 through the shaft 202.

In other aspects, as further shown in FIG. 5, the medical system 200 may include a controller 209 operably coupled to the delivery assembly 201 and/or the shaft 202. For instance, the controller 209 may be operably coupled to the first container 203, the second container 205, and/or the third container 207 of the delivery assembly 201. The controller 209 may be configured to control flow of one or more substances from the first container 203, the second container 205, and/or the third container 207. For instance, in some examples, the controller 209 may be configured to transmit a first volume of first substance from the first container 203, and a second volume of second substance from the second container 205 through the shaft 202. In another example, the controller 209 may be configured to control gas pressure to transmit a third volume of third substance from the third container 207 through the shaft 202. The controller 209 may be able to precisely control the volume of substance and/or duration of transmission of substances from the delivery assembly 201. For example, the controller 209 may control movement and/or force on plungers of syringes of the first container 203, the second container 205, and/or the third container 207.

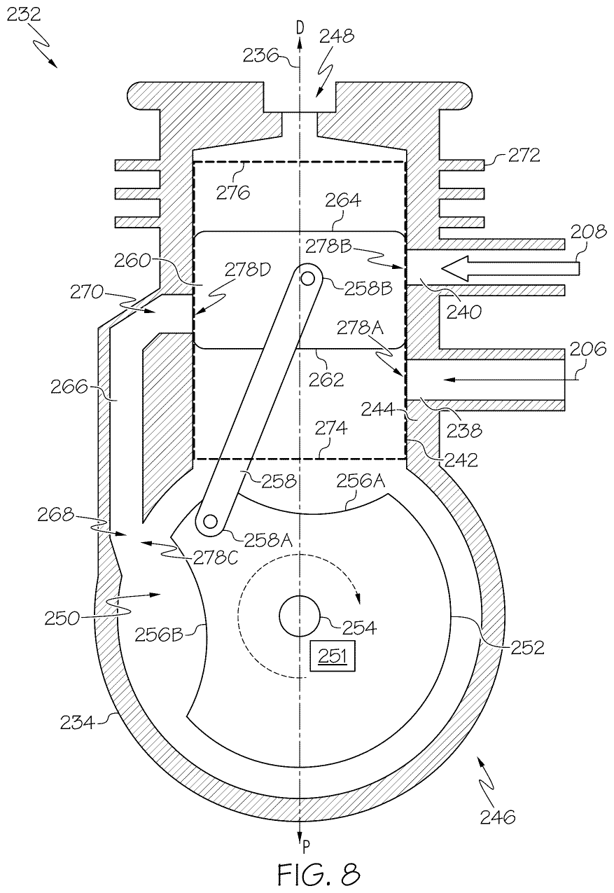

In some implementations, the controller 209 is coupled to the mixer assembly 232. The controller 209 may be electrically coupled to the mixer assembly 232 (e.g., transmit one or more electrical signals corresponding to operation instructions for one or more portions of mixer assembly 232). As discussed in detail below, mixer assembly 232 may include a driver 250, a movable portion 252, and a piston 260 (FIG. 8). In some aspects, the controller 209 may be configured to control motion of the movable portion 252 relative to the mixing chamber 242, and thereby control motion of the piston 260 through the mixing chamber 242. The controller 209 and/or the mixer assembly 232 may include additional electronic components (e.g., circuit board, wires, resistors, capacitors, power source, etc.) to provide or facilitate this electrical communication. In some implementations, controller 209 may be mechanically coupled to mixer assembly 232, such as, e.g., via a plurality of articulating links extending distally from a handle (not shown) and movably coupled to the driver 250. For instance, the handle may include one or more movable actuators (e.g., button, wheel, etc.) movably coupled to the plurality of articulating links, such that actuation of the movable actuator causes motion of the driver 250.

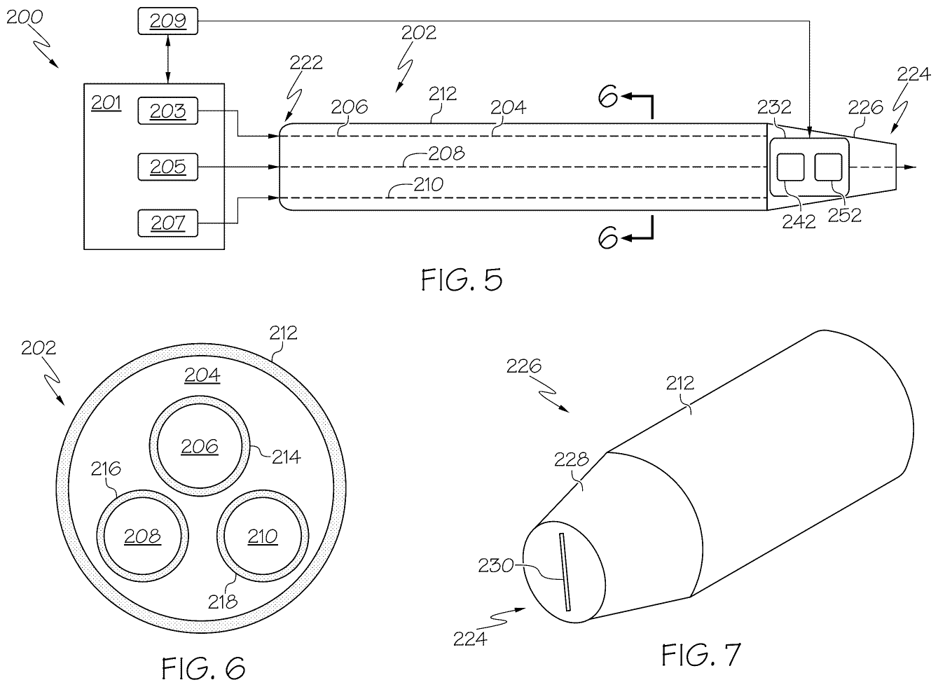

FIG. 6 shows a cross-section (e.g., lateral cross-section) of the shaft 202 along a line 6-6 shown in FIG. 5, according to aspects of the disclosure. The plurality of lumens of the shaft 202 may include a central lumen 204, first lumen 206, second lumen 208 and/or third lumen 210. As discussed herein, the first lumen 206, the second lumen 208, and/or the third lumen 210 may be in fluid communication with a mixer assembly 232 disposed in the distal portion of the shaft 202. For example, the mixer assembly 232 may be disposed in a distal tip 226 of the shaft 202. The central lumen 204 is defined by an outer wall 212 which surrounds the first lumen 206, second lumen 208, and third lumen 210. The first lumen 206 is defined by a first inner wall 214 disposed within the central lumen 204. The second lumen 208 is defined by a second inner wall 216 disposed within the central lumen 204. The third lumen 210 is defined by a third inner wall 218 disposed within the central lumen 204. The first inner wall 214, the second inner wall 216, and/or the third inner wall 218 may be secured to the outer wall 212, for example, by one or more protrusions to maintain spacing between respective lumens relative to central lumen 204. Further, the first inner wall 214, the second inner wall 216, and/or the third inner wall 218 may be secured to another one of the first inner wall 214, the second inner wall 216, and/or the third inner wall 218. Alternatively or additionally, the first inner wall 214, the second inner wall 216, and/or the third inner wall 218 may be secured to the outer wall 212. In some aspects, the shaft 202 does not include central lumen 204, for example, with the first inner wall 214, the second inner wall 216, and/or the third inner wall 218 being formed in shaft 202 (e.g., via an extrusion).

FIG. 7 shows a perspective view of the distal tip 226, according to aspects of the disclosure. Distal tip 226 may be integrally formed with the shaft 202, or may be attached to the distal portion of the shaft 202 by, for example, an adhesive, a snap-fit connection, threaded connection, or the like. The distal tip 226 may include an attachment feature (not shown), such as an internal thread or latch, configured for securing the distal tip 226 to and/or radially around the mixer assembly 232. For instance, the distal tip 226 may include one or more attachment features configured to engage and couple to an external feature 272 of the mixer assembly 232, such as internal thread configured to threadably engage external thread. It should be understood that the attachment feature may additionally or alternatively include other mechanisms to secure the distal tip 226 and mixer assembly 232, such as a latch or lock. Distal tip 226 may have variable dimensions at different portions along a length thereof, for example a distally tapering portion 228. Distal tip 226 may include a distal wall positioned at the distal end 224 of the shaft 202 and having an opening 230 (e.g., a slit that is biased toward a closed configuration) formed therein. As shown and discussed herein, the distal tip 226 may be in fluid communication with the mixer assembly 232. For example, distal tip 226 may include a duckbill nozzle coupled to the mixer assembly 232 and used to expel a mixture of substances from the mixer assembly 232 (e.g., through the opening 230 at the distal end 224).

FIG. 8 shows the mixer assembly 232 of medical system 200, according to aspects of the disclosure. The mixer assembly 232 may be configured to receive and mix a plurality of substances therein, and to displace the mixture of substances through one or more openings formed therein. As shown, the mixer assembly 232 includes a body 234, which may be formed of any material or combination of materials capable of containing components of the mixer assembly 232 discussed herein. The body 234 may define a mixing chamber 242 therein, extending between a proximal portion 246 and a distal portion 248 of the mixing chamber 242 along a longitudinal axis 236 of the mixer assembly 232.

The mixer assembly 232 may include one or more ports extending through one or more walls of the body 234 and into the mixing chamber 242. The one or more ports may fluidly couple one or more lumens of the shaft 202 and the mixer assembly 232, such that the mixing chamber 242 may receive one or more substances from the shaft 202 therein. For example, mixer assembly 232 may include a first port 238 and a second port 240, respectively extending through a first wall 244 of the body 234 into mixing chamber 242. As shown, first port 238 extends through the first wall 244 at a first location 278A relative to longitudinal axis 236, and second port 240 extends through the first wall 244 at a second location 278B different than the first location 278A relative to a longitudinal axis 236 of the mixing chamber 242 (e.g., second location 278B may be distal of first location 278A relative to longitudinal axis 236). However, it should be understood that the mixer assembly 232 may additionally or alternatively include one or more other ports, for example, which may help to enable fluidic connection(s) or coupling to one or more medical devices and components thereof.

As further shown, the mixer assembly 232 includes a driver 250 configured to drive motion of one or more components relative to the mixing chamber 242. The driver 250 may be a drive assembly including two or more components which move within and relative to the mixing chamber 242. The driver 250 may include a movable portion 252 disposed in the proximal portion 246 of the mixing chamber 242. The movable portion 252 may move relative to the mixing chamber 242 (e.g., rotate, pivot, etc.). For instance, the movable portion 252 may rotate ±360° about a pivot point 254. In some implementations, the driver 250 includes one or more motors coupled, directly or indirectly, to the movable portion 252. For instance, in some examples, the driver 250 may include a motor 251, such as a two-stroke motor and/or electric motor. The driver 250 may include a drive shaft extending from the motor 251 (e.g., at the pivot point 254), and the movable portion 252 may include a rotor or rotatable member disposed on a distal end of the drive shaft (e.g., to rotate about the pivot point 254).

The movable portion 252 may include an outer surface having one or more grooves, indentations, collection ducts, surface features, etc. formed therein. As the movable portion 252 moves about the pivot point 254, the outer surface of movable portion 252 may help to displace one or more substances relative to the mixing chamber 242. For example, as shown in FIG. 8, the movable portion 252 may include at least a first groove 256A and a second groove 256B. The first groove 256A and second groove 256B may help to displace one or more substances within proximal portion 246 of the mixing chamber 242. As the movable portion 252 moves about the pivot point 254 in a first direction (e.g., clockwise or counter-clockwise), the first groove 256A and/or second groove 256B may help to displace the substance(s) flowing through the first port 238 and/or the second port 240 in the first direction along a path defined between the movable portion 252 and inner surface of the body 234. In some implementations, the first groove 256A and/or the second groove 256B are configured to collect a volume of the substance within the mixing chamber 242. For example, when the movable portion 252 is in a first position relative to the mixing chamber 242, the first groove 256A may collect a volume of the first substance flowing through the first port 238. Furthermore, when the movable portion 252 moves to a second position different than the first position relative to the mixing chamber 242, the first groove 256A may transmit, deposit, or otherwise urge the volume of the first substance to another location. As shown, each of the first groove 256A and second groove 256B have concave geometry; however, it should be understood that the movable portion 252 may additionally or alternatively include other grooves, indentations, ducts, surface features, etc. having different shapes and dimensions, such as convex extensions, bumps, circular holes, etc.

The mixer assembly 232 may further include a piston 260 movably disposed in the mixing chamber 242. The piston 260 may include a distal surface 264 oriented toward the distal portion 248 of the mixing chamber 242, and a proximal surface 262 oriented toward the proximal portion 246 of the mixing chamber 242. The piston 260 may move within the mixing chamber 242 (e.g., translate) between different positions corresponding to different locations along the longitudinal axis 236. The piston 260 may proximally and distally translate through the mixing chamber 242 between a proximalmost position 274 and a distalmost position 276. The piston 260 may proximally translate such that the proximal surface 262 is positioned at the proximalmost position 274, and distally translate such that the distal surface 264 is positioned at the distalmost position 276. The driver 250 may include an arm 258 having a first end 258A coupled to the movable portion 252, and a second end 258B coupled to the piston 260 opposite the first end 258A of arm 258. The motor 251 may be configured to move the movable portion 252 about the pivot point 254, causing the arm 258 to move relative to the mixing chamber 242, and in turn causing the piston 260 to move (e.g., move longitudinally) relative to the mixing chamber 242. The motor 251 may be positioned away from the movable portion 252 and the pivot point 254, such that rotation of the movable portion 252 causes the longitudinal translation of the arm 258 and the piston 260. For example, coupling via the arm 258 may cause distal or proximal translation of the piston 260 through the mixing chamber 242 along the longitudinal axis 236.

As the piston 260 moves through the mixing chamber 242, at least one of the distal surface 264 and/or proximal surface 262 may be in fluid communication with one or more substances flowing into the mixing chamber 242, for example, to help displace the one or more substances to one or more locations of the mixing chamber 242. For example, the piston 260 may expel a plurality of substances (e.g., mixed substances) disposed on the distal surface 264 from the mixing chamber 242 (e.g., in the distalmost position 276). In some positions along the longitudinal axis 236, one or more surfaces of the piston 260 (e.g., sidewalls between the proximal surface 262 and the distal surface 264) may block or otherwise inhibit one or more substances flowing into the mixing chamber 242. For example, one or more sidewalls of the piston 260 may align at least partially with the first port 238 and/or the second port 240, which at least partially hinders or impedes flow of respective substances through the first port 238 and/or the second port 240 into the mixing chamber 242.

The position of piston 260 may determine whether the proximal surface 262 and/or the distal surface 264 is in fluid communication or abutting contact with one or more substances. In some positions along the longitudinal axis 236, the proximal surface 262 of the piston 260 is in fluid communication with the first port 238, for example, to help displace the first substance toward the proximal portion 246 of the mixing chamber 242. In some positions along the longitudinal axis 236, the distal surface 264 of the piston 260 is in fluid communication with the second port 240, for example, to displace the second substance toward the distal portion 248 of the mixing chamber 242. In some positions along the longitudinal axis 236, the distal surface 264 of the piston 260 is positioned to receive a first volume of first substance and a second volume of second substance thereon. As discussed herein, the first volume of first substance may flow through a transfer conduit 266 and the second volume of second substance may flow through second port 240.

As further shown in FIG. 8, the transfer conduit 266 may extend between a first end 268 and a second end 270. The first end 268 having a first opening (e.g., proximal opening) in fluid communication with a third location 278C of the mixing chamber 242, and the second end 270 having a second opening (e.g., distal opening) in fluid communication with a fourth location 278D of the mixing chamber 242. The third location 278C may be positioned in the proximal portion 246 of mixing chamber 242, such that first end 268 of transfer conduit 266 is in fluid communication with proximal portion 246 and adjacent to the movable portion 252 of the driver 250. The fourth location 278D may be positioned in the mixing chamber 242 between the proximal portion 246 and distal portion 248 (e.g., a middle or intermediate portion), for example, positioned distal of the third location 278C relative to the longitudinal axis 236. For instance, as the first substance flows through the first port 238 into the mixing chamber 242, the movable portion 252 may help to displace the first substance through the first end 268 of the transfer conduit 266. As more of the first substance is displaced through first end 268, the first substance flows distally through the transfer conduit 266 towards the second end 270 and into the mixing chamber 242 at the fourth location 278D. The transfer conduit 266 therefore may include a proximal opening at the first end 268 positioned adjacent to the movable portion 252 (e.g., at the third location 278C), and a distal opening at the second end 270 spaced distally from the proximal opening (e.g., at the fourth location 278D). The distal opening at the second end 270, for example, positioned distal of the first port 238 and proximal of the second port 240 relative to the longitudinal axis 236. Although shown as a single opening at each of the first end 268 and the second end 270, it should be understood that the transfer conduit 266 may include a plurality of openings positioned at different locations. For example, the transfer conduit 266 may include a plurality of openings disposed circumferentially about the longitudinal axis 236 and in fluid communication with the mixing chamber 242.

In some implementations, the first groove 256A and/or second groove 256B may help to displace one or more substances through and/or toward first end 268 of the transfer conduit 266. For instance, as the movable portion 252 moves relative to the mixing chamber 242, the first groove 256A and/or the second groove 256B may collect a volume of the first substance therein. As the movable portion 252 continues to move relative to the mixing chamber 242, the first groove 256A and/or the second groove 256B may help to deposit or transmit the volume of the first substance through the first end 268 of the transfer conduit 266. As more of the first substance is displaced through first end 268, the first substance moves through transfer conduit 266 and through the second end 270 into the mixing chamber 242 at the fourth location 278D.

The piston 260 may proximally translate through the mixing chamber 242 and in turn urge the first substance through the transfer conduit 266. For example, proximal translation of the piston 260 may cause the proximal surface 262 to displace the first substance proximal of the piston 260, thereby reducing a volume of space in the proximal portion 246 of the mixing chamber 242 and pushing some of the first substance toward and/or through the transfer conduit 266. As the first substance accumulates in the transfer conduit 266, a volume of first substance is displaced through the second end 270 of the transfer conduit 266. The second end 270 of the transfer conduit 266 may be proximal of the second port 240 on an opposing wall of the mixing chamber 242 at the fourth location 278D. As the first substance flows into the mixing chamber 242, first substance may contact second substance flowing through the second port 240. For example, the first substance and second substance may mix on the distal surface 264 of the piston 260. In turn, the piston 260 may distally translate through the mixing chamber 242 and dispense the mixture of substances on the distal surface 264 (e.g., through the distal tip 226).

As shown, the outer surface of body 234 may include the external feature 272 disposed thereon (e.g., external thread, latch, lock, etc.). The external feature 272 may be used to couple the distal tip 226 thereon. In other implementations, the distal tip 226 is integrally formed, or otherwise permanently coupled, on mixer assembly 232.

Aspects of any of the aforementioned systems, devices, assemblies, and methods may be used in any combination or arrangement. For example, the mixer 118 of FIG. 1 may be used in combination with the mixer assembly 232 of FIG. 5. Additional combinations or arrangements are also contemplated herein. Any embodiment discussed herein may include the distal tip 226 and/or the mixer assembly 232. For example, to allow the mixed substance to be delivered distally while helping prevent substances and/or other fluid(s) from the target treatment site flowing proximally into the distal end of the medical device.

Each of the aforementioned systems, devices, assemblies, and methods may be used to help protect and/or treat target treatment sites by delivering a mixture of substances (e.g., a gel, a liquid, a powder, prophylactic agents, etc.) to the target treatment site. By providing a medical device with multiple lumens for delivering the mixture of substances, known problems associated with premature curing within the medical device and/or other aspects of invasive surgical procedures may be reduced or avoided. For example, mixing two or more substances in a distal portion of the medical device prior to delivery at the target treatment site may help to mitigate the formation of clogs or other obstructions in the medical device due to premature curing of the mixture of substance. Further, mitigating formation of clogs or other obstructions may reduce preparation and/or procedure time for the medical procedure and/or maintenance of the medical device. Accordingly, physicians or other users of may reduce the overall procedure time, increase efficiency of procedures, and/or avoid unnecessary harm to a subject's body due to limited ability to deliver the mixture of substances to the target treatment site.

It will be apparent to those skilled in the art that various modifications and variations may be made in the disclosed systems, devices, assemblies, and methods without departing from the scope of the disclosure. Other aspects of the disclosure will be apparent to those skilled in the art from consideration of the specification and practice of the features disclosed herein. It is intended that the specification and examples be considered as exemplary only, with a true scope of the disclosure being indicated by the following claims and their equivalents.

We claim:

1. A medical device, comprising:
a shaft including a plurality of lumens, each of the plurality of lumens including an inlet at a proximal portion of the shaft and an outlet at a distal portion of the shaft; and
a mixer assembly disposed in the distal portion of the shaft and in fluid communication with the plurality of lumens, wherein the mixer assembly comprises:
a mixing chamber configured to receive a plurality of substances from the plurality of lumens;
a piston movably disposed in the mixing chamber; and
a driver operably coupled to the piston,
wherein the driver is configured to drive the piston relative to the shaft within the mixing chamber, and in turn expel the plurality of substances from the mixing chamber.

2. The medical device of claim 1, wherein the mixer assembly comprises a distal tip in fluid communication with a distal end of the mixing chamber.

3. The medical device of claim 2, wherein the distal tip is a duckbill nozzle configured to expel the plurality of substances through an opening formed therein.

4. The medical device of claim 2, wherein the mixing chamber comprises an outer surface having an external thread disposed thereon, and wherein the distal tip comprises an internal thread configured to threadably engage the external thread.

5. The medical device of claim 1, wherein the driver comprises a movable portion configured to move within the mixing chamber, and an arm coupled to the movable portion and the piston.

6. The medical device of claim 5, wherein the mixer assembly comprises a plurality of ports in fluid communication with the plurality of lumens and the mixing chamber.

7. The medical device of claim 6, wherein the plurality of ports comprises:
a first port extending into the mixing chamber at a first location relative to a longitudinal axis of the mixing chamber; and
a second port extending into the mixing chamber at a second location relative to the longitudinal axis,
wherein the first location is positioned proximal of the second location and distal of the movable portion of the driver relative to the longitudinal axis.

8. The medical device of claim 7, wherein the mixer assembly comprises:
a transfer conduit including a first end in fluid communication with a third location of the mixing chamber, and a second end in fluid communication with a fourth location of the mixing chamber;
wherein the third location is positioned adjacent to the movable portion of the driver and proximal of the first location relative to the longitudinal axis, and
wherein the fourth location is positioned distal of the first location and proximal of the second location relative to the longitudinal axis.

9. The medical device of claim 8, wherein the movable portion of the driver comprises at least one groove configured to displace a volume of at least one substance of the plurality of substances from the mixing chamber toward the first end of the transfer conduit.

10. The medical device of claim 7, wherein the first location is positioned proximal of a distalmost position of the piston, and wherein the second location is positioned distal of a proximalmost position of the piston.

11. The medical device of claim 5, wherein the movable portion is configured to move within the mixing chamber about a pivot point, which in turn translates the arm proximally and/or distally relative to the longitudinal axis, and in turn translates the piston longitudinally proximally and/or distally within the mixing chamber along the longitudinal axis.

12. The medical device of claim 11, wherein the driver comprises a motor coupled to the movable portion, wherein the motor is configured to rotate the movable portion about the pivot point.

13. The medical device of claim 12, wherein the motor comprises a two-stroke motor.

14. The medical device of claim 1, wherein the plurality of lumens includes a first lumen and a second lumen, wherein the plurality of substances that the first lumen and the second lumen are configured to deliver includes a liquid or gel substance and an accelerant substance different from the liquid or gel substance, wherein the first lumen is configured to deliver the liquid or gel substance into the mixing chamber, and wherein the second lumen is configured to deliver the accelerant substance into the mixing chamber.

15. The medical device of claim 14, wherein the first lumen and the second lumen are fluidly isolated from each other between a proximal end of the shaft and the mixer assembly.

16. A medical system, comprising:
a delivery assembly configured to deliver two or more substances;
a medical device in fluid communication with the delivery assembly, wherein the medical device comprises:
a shaft including a plurality of lumens, each lumen of the plurality of lumens including an inlet at a proximal portion of the shaft and a distal opening at a distal portion of the shaft; and
a mixer assembly disposed in the distal portion of the shaft and in fluid communication with the plurality of lumens, wherein the mixer assembly comprises:
a mixing chamber configured to receive a plurality of substances from the delivery assembly through the plurality of lumens;
a piston movably disposed in the mixing chamber; and
a driver including a movable portion coupled to the piston,
wherein the movable portion is configured to drive the piston relative to the shaft within the mixing chamber, and in turn expel the plurality of substances from the mixing chamber.

17. The medical system of claim 16, further comprising a controller electrically coupled to the mixer assembly, wherein the controller is configured to control motion of the driver relative to the mixing chamber, and thereby to control motion of the piston through the mixing chamber.

18. The medical system of claim 16, wherein the movable portion includes an outer surface having at least one groove formed therein, wherein the groove is configured to receive a volume of at least one of the plurality of substances therein, and in turn to displace the volume as the movable portion moves relative to the mixing chamber.

19. A medical system comprising:
a first syringe configured to contain and provide a first substance;
a second syringe configured to contain and provide a second substance;
a shaft including a first lumen in fluid communication with the first syringe, a second lumen in fluid communication with the second syringe, and a nozzle at a distal end of the shaft; and
a mixer assembly disposed in a distal portion of the shaft, wherein the mixer assembly comprises:
a mixing chamber in fluid communication with the first lumen and the second lumen, wherein the mixing chamber is fluidly connected to the nozzle;
a piston movably disposed in the mixing chamber; and
a driver coupled to the piston,
wherein the driver comprises a motor configured to move the piston through at least a portion of the mixing chamber from a first position in a proximal portion to a second position at a distal portion.

20. The medical system of claim 19, wherein the mixer assembly comprises:
a first port in fluid communication with the mixing chamber and a distal opening of the first lumen;
a second port in fluid communication with the mixing chamber and a distal opening of the second lumen;
a transfer conduit, wherein the transfer conduit includes a proximal opening adjacent to a movable portion of the driver, and wherein the transfer conduit includes a distal opening spaced distally from the proximal opening; and
wherein the movable portion of the driver is configured to displace a volume of at least one of the first substance and the second substance through the proximal opening of the transfer conduit;
wherein the distal opening of the transfer conduit is positioned across from the first port and the second port relative to a longitudinal axis of the mixing chamber, and wherein the distal opening is positioned distal of at least one of the first port and the second port.
